(19) United States
(12) Patent Application Publication
(10) Pub. No.: US 2014/0343076 A1
(43) Pub. Date: Nov. 20, 2014

(54) PHARMACEUTICAL COMPOSITIONS OF LURASIDONE

(71) Applicant: Cadila Healthcare Limited, Ahmedabad (IN)

(72) Inventors: Sushrut Krishnaji Kulkarni, Gujarat (IN); Ajay Kumar Handa, Gujarat (IN); Doss Pothuvan Uma, Gujarat (IN); Yagnesh Atulkumar Bhatt, Gujarat (IN)

(73) Assignee: Cadila Healthcare Limited, Ahmedabad (IN)

(21) Appl. No.: 14/278,874

(22) Filed: May 15, 2014

(30) Foreign Application Priority Data
May 17, 2013 (IN) 1768/MUM/2013

(51) Int. Cl. A61K 31/496; A61K 45/06; A61K 31/194

(52) U.S. Cl. CPC A61K 31/496; A61K 31/194; A61K 45/06
USPC 514/254.04

(57) ABSTRACT

The present invention relates to pharmaceutical compositions of lurasidone or salts thereof. In particular, the invention relates to pharmaceutical compositions of lurasidone or salts thereof with one or more acids. The invention also relates to processes for the preparation of such compositions and use thereof for treatment of schizophrenia, bipolar disorders or senile dementia.

### FIELD OF THE INVENTION

This application claims the benefit of priority, under 35 U.S.C. Section 119, to India Patent Application Serial No. 1768/MUM/2013, filed May 17, 2013, which is incorporated herein by reference in its entirety.

### FIELD OF THE INVENTION

The present invention relates to pharmaceutical compositions of lurasidone or salts thereof. In particular, the invention relates to pharmaceutical compositions of lurasidone or salts thereof with one or more acids. The invention also relates to processes for the preparation of such compositions and use thereof for treatment of schizophrenia, bipolar disorders or senile dementia.

### BACKGROUND OF THE INVENTION

Lurasidone is a well-known dopaminergic (D2) and serotonin (5-HT2A) receptor antagonist and is disclosed in U.S. Pat. No. 5,532,372. Chemically, it is N-[4-[4-(1,2-benzisothiazol-3-yl)-1-piperazinyl]-(2R,3R)-2,3-tetramethylene-butyl]-(1′R,2′S,3′R,4′S)-2,3-bicyclo[2,2,1]heptanedicarboxyimide hydrochloride having the structural formula

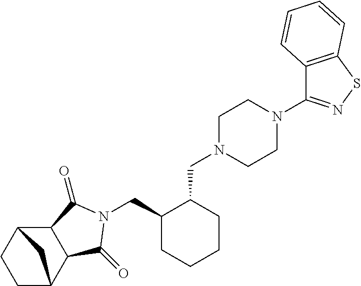 [SMILES: O=C1[C@@H]2[C@H]3CC[C@H](C3)[C@@H]2C(=O)N1C[C@@H]1CCCC[C@H]1CN1CCN(c2nsc3ccccc23)CC1]

A free form of lurasidone and an acid addition salt thereof are known to have psychotropic activities and are effective as therapeutic agents, particularly for schizophrenia or senile dementia, etc. Senile dementia is broadly classified into Alzheimer's dementia and cerebrovascular dementia, and it can be said that the two make up about 80% of senile dementia.

U.S. Pat. No. 7,727,553 discloses a pharmaceutical preparation in the form of a rapidly disintegrating oral preparation comprising lurasidone, two disintegrating agents, a water-soluble excipient and a water soluble polymer binder.

U.S. Publication No. 2010/0093875 discloses a granular preparation with suppressed caking which comprises lurasidone, sugar or sugar alcohols, organic acids and pullulan or dextrin.

U.S. Publication No. 2009/0286805 discloses a solution-type preparation of lurasidone or acid addition salt thereof prepared by incorporating one or more substances selected from benzyl alcohol, N,N-dimethylacetamide, lactic acid, anhydrous ethanol and propylene glycol.

U.S. Publication No. 2009/0143404 discloses a composition comprises lurasidone, pregelatinized starch, a water-soluble excipient and water-soluble polymer binder. It also discloses lurasidone in an amount of 20-45% w/w.

In order to secure the bioequivalence when a pharmaceutical preparation having different amounts is administered at the same dose, there was issued “Guideline for Bioequivalence testing of Oral Solid Dosage Forms with Different Content” (Notification No. 64 of the Evaluation and Licensing Division, PMSD dated Feb. 14, 2000), by which it has been required that a pharmaceutical preparation having different amounts should be equivalent in dissolution profile in test solutions such as buffers of pH 1.2, 3.0 to 5.0 and 6.8 (which correspond to the pH values of the stomach, the intestine and the oral cavity, respectively), water, and saline solution, etc.

For medicaments showing a good solubility in water, it is easy to prepare such preparations having equivalent in vitro (dissolution) profile even in different amounts due to their water solubility. On the contrary, for medicaments containing as an active ingredient a slightly water-soluble compound, such as lurasidone (has a solubility of less than several μg/ml in water), it is difficult to prepare a pharmaceutical preparation having equivalent dissolution profile, and even more challenging to have such equivalent in vitro (dissolution) profile over a wide range of medicament content.

The prior art references emphasize on using water-soluble excipients, for example—water-soluble diluents, water-soluble polymer binders, extragranular disintegrants and there is no disclosure or teaching/suggestion in the art about how to develop stable formulations of lurasidone using acids which can also exhibit rapid disintegration as well as equivalent in vitro (dissolution) profile over wide dose range.

Hence, there still remains a need for alternative pharmaceutical compositions comprising lurasidone in order to achieve desired dissolution profile of the compositions containing wide dose of active ingredient.

### SUMMARY OF THE INVENTION

In one general aspect there is provided a pharmaceutical composition comprising lurasidone or salts thereof, at least one acid and one or more pharmaceutically acceptable excipients.

In another general aspect there is provided a solid dosage form comprising lurasidone or salts thereof, at least one acid and one or more pharmaceutically acceptable excipients.

In another general aspect there is provided a pharmaceutical composition comprising lurasidone or salts thereof, at least one acid, at least one surfactant and one or more pharmaceutically acceptable excipients.

In another general aspect there is provided a solid dosage form comprising lurasidone or salts thereof, an organic acid, a surfactant and one or more pharmaceutically acceptable excipients.

In another general aspect there is provided an immediate-release pharmaceutical composition comprising lurasidone or salts thereof, at least one acid and one or more pharmaceutically acceptable excipients.

In another general aspect there is provided a pharmaceutical composition of lurasidone or salts or enantiomer thereof, comprising one or more acids, one or more disintegrating agents, diluents, fillers, binders, surfactants, lubricants, glidants, sweeteners and flavoring agents.

In another general aspect there is provided a stable pharmaceutical composition comprises lurasidone or salts thereof, at least one acid, at least one surfactant and one or more pharmaceutical excipients and retains at least 80% of the potency of lurasidone or salts thereof in the pharmaceutical composition after storage at 40° C. and 75% relative humidity for three months.

In another general aspect there is provided a process for preparing a pharmaceutical composition of lurasidone or salts thereof. The process comprises the steps of admixing and/or granulating lurasidone or salts thereof with at least one acid and one or more pharmaceutically acceptable excipients and processing the mixture to provide a final dosage form.

In another general aspect there is provided a method of treating schizophrenia, bipolar disorder or senile dementia in patient comprising administering to said subject a pharmaceutical composition comprising lurasidone or salts thereof, at least one acid and one or more pharmaceutically acceptable excipients.

The details of one or more embodiments of the present invention are set forth in the description below. Other features, objects and advantages of the invention will be apparent from the description.

### DETAILED DESCRIPTION OF THE INVENTION

We have surprisingly found that compositions of lurasidone can be prepared using at least one acid and such formulations have a similar in vitro (dissolution) profile to Latuda®, marketed form of lurasidone in USA.

The inventors have developed pharmaceutical compositions of lurasidone using at least one acid. In particular, the inventors have developed pharmaceutical compositions by careful selection of acids with their optimum concentrations.

Moreover, such formulations are also stable and may retain at least 80% of the potency of lurasidone or salts thereof in the pharmaceutical composition after storage at 40° C. and 75% relative humidity for three months.

As used herein, the term ‘lurasidone’ is used in broad sense to include not only the lurasidone per se but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof, and also its various crystalline and amorphous forms.

The phrase “pharmaceutically acceptable” is employed herein to refer to those compounds, materials, compositions; and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, the term “acid” includes organic acids and inorganic acids those known to person skilled in the art, preferably, acids suitable to achieve the equivalent dissolution profile of lurasidone compositions of wide dose range (e.g. 5 to 120 mg). Non limiting examples of such organic acids are—citric acid, malic acid, tartaric acid, fumaric acid, succinic acid, ascorbic acid and hydrates thereof. Non-limiting examples of inorganic acids are—hydrochloric acid, boric acid, sulfuric acid, phosphoric acid and hydrates thereof. Said organic and inorganic acids may be used alone, or two or more thereof may be used together.

Embodiments of the present invention relate to pharmaceutical compositions of lurasidone or salts thereof, at least one acid and one or more pharmaceutically acceptable excipients.

The pharmaceutical composition of the present invention refers to pharmaceutical compositions which can be formulated into powder, granule, fine granule/micro-granules, pellets, wafers, tablet or capsule.

In one embodiment, the pharmaceutical composition comprises lurasidone or salts thereof, at least one organic acid and one or more pharmaceutically acceptable excipients.

In another embodiment, the pharmaceutical composition comprises lurasidone or salts thereof, at least one inorganic acid and one or more pharmaceutically acceptable excipients.

In another embodiment, the pharmaceutical composition comprises lurasidone or salts thereof, combination of organic and inorganic acids and one or more pharmaceutically acceptable excipients.

In another embodiment, the pharmaceutical composition comprises lurasidone or salts thereof, at least one acid and one or more pharmaceutically acceptable excipients wherein, the amount of acid is in the range of about 0.1 to 10% by weight (% w/w, hereinafter the same) to the weight of composition, preferably 3-7% by weight of the composition.

In another embodiment, the pharmaceutical compositions of lurasidone optionally have functional as well non-function coating. The functional coatings may include controlled-release and/or delayed release coating and non-functional coating may include seal coatings and/or elegant coatings.

In another embodiment, pharmaceutical composition comprising non-pareil seeds or sugar spheres or neutral excipients coated with one or more drug layers comprising lurasidone or salts thereof with one or more acids. The multiple unit cores may be further coated with one or more layers of natural or synthetic polymers.

In another embodiment, a stable pharmaceutical composition comprises lurasidone or salts thereof, at least one acid, at least one surfactant and one or more pharmaceutical excipients and retains at least 80% of the potency of lurasidone or salts thereof in the pharmaceutical composition after storage at 40° C. and 75% relative humidity for three months.

In another embodiment, the pharmaceutical composition comprises a matrix comprising lurasidone or salts thereof and one or more acids. Alternatively, said matrix may be coated further with one or more layers of natural or synthetic polymers.

In another embodiment, the desired dissolution profile of the pharmaceutical composition according to the present invention can be achieved by using acid in the range of about 0.1 to 10% by weight to the total weight of composition.

In another embodiment, the pharmaceutical composition may be provided as a tablet, which may be film coated with one or more coating agents or coated with release rate-controlling polymers. The coating agents may include hypromellose, polyvinyl alcohol, and sodium carboxymethyl cellulose. The rate-controlling polymers include, but not limited to, polymers or copolymers of methacrylic acid and phthalate. The coating may also include one or more plasticizers, pigments, pore forming materials or suspension stabilizers, such as polyethylene glycol, talcum or titanium dioxide.

The pharmaceutically acceptable excipients for use in the pharmaceutical composition of lurasidone may include one or more diluents, fillers/bulking agents, binders, disintegrants, surfactants, lubricants, glidants, sweeteners/taste masking agents, colorants and flavors.

Suitable diluents/fillers or bulking agents include, but are not limited to, microcrystalline cellulose, di- or tri-basic calcium phosphate, crystalline cellulose, powdered cellulose, calcium carbonate, calcium sulphate, magnesium silicate, magnesium trisilicate, magnesium aluminium metasilicate (Neusilin), kaolin, starch, starch derivatives, magnesium carbonate, magnesium oxide and co-processed insoluble excipients.

Suitable disintegrants include, but are not limited to, Veegum (highly refined isomorphous silicate), crospovidone, cellulose, kaolin, crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. Preferred disintegrants among these disintegrants include crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. The amount of disintegrant in the pharmaceutical composition ranges from about 0.5% to about 10% by total weight of the composition.

Suitable surfactants include, but are not limited to, anionic, cationic, non-ionic or amphoteric surfactants or those known to the person skilled in the art. The amount of surfactant present in the pharmaceutical composition of lurasidone, its enantiomer or salt thereof ranges from about 0.5% to about 25% by total weight of the composition.

Suitable lubricants and glidants include, but are not limited to, stearic acid and its derivatives or esters like sodium stearate, magnesium stearate and calcium stearate and the corresponding esters such as sodium stearyl fumarate; talc and colloidal silicon dioxide.

Suitable taste masking agents include, but are not limited to, one or more of polymers, surfactants, sweeteners and flavors. Examples of polymers include one or more of cellulose acetate, polymethacrylates, cellulose derivatives such as hydroxypropylmethyl cellulose, hydroxypropyl cellulose, hydroxylethyl cellulose; and the like. Examples of sweeteners include but not limiting to one or more of aspartame, saccharin, sucralose, glycyrrhizin; and the like.

Suitable sweeteners include, but are not limited to, saccharides such as aspartame, sugar derivatives. Other examples of sweeteners comprise sodium saccharin; aspartame; sugarless sweeteners, hydrogenated starch hydrolysates, alone or in combination.

Suitable flavors include, but are not limited to, cinnamon, wintergreen, eucalyptus, spearmint, peppermint, menthol, anise as well as fruit flavors such as apple, pear, peach, vanilla, strawberry, cherry, apricot, orange, watermelon, banana and the like; bean-derived flavors, such as coffee, cocoa and the like or mixtures thereof.

In another embodiment, the pharmaceutical composition of lurasidone or salts thereof may be prepared by any suitable method known in the art such as direct compression, dry or wet granulation, fluidized bed granulation, melt extrusion, melt granulation, spray coating, freeze drying, spray drying and solution evaporation.

In another embodiment, the pharmaceutical composition of lurasidone or salts thereof is prepared by

(1) premixing the lurasidone or salts thereof and the main portion of the excipients including the binder to obtain a pre-mixture;
(2) granulating the pre-mixture by adding the granulation liquid, preferably water;
(3) drying the granules in a fluidized bed dryer or a drying oven;
(4) optionally dry sieving of the dried granules;
(5) mixing the dried granules with the remaining excipients like a glidant and a lubricant to obtain the final mixture;
(6) tableting the final mixture by compressing it on a suitable tablet press to produce tablets cores; and
(7) optionally film-coating of the tablet cores with a film coat.

In another embodiment, the pharmaceutical composition of lurasidone or salts thereof is prepared by

(1) premixing lurasidone or salts thereof and the main portion of the excipients to obtain a pre-mixture;
(2) optionally dry screening the pre-mixture through a screen in order to segregate cohesive particles and to improve content uniformity;
(3) mixing the pre-mixture of step (1) or (2) in a mixer, optionally by adding remaining excipients to the mixture and continuing mixing;
(4) tableting the final mixture of step (3) by compressing it on a suitable tablet press to produce the tablet cores; and
(5) optionally film-coating of the tablet cores of step (4) with a film coat.

In another embodiment, the pharmaceutical composition of lurasidone or salts thereof is prepared by

(1) mixing lurasidone or salts thereof with either all or a portion of the excipients;
(2) compaction of the mixture of step (1) on a suitable roller compactor;
(3) reducing the ribbons obtained during step (2) to granules by suitable milling or sieving steps;
(4) optionally mixing the granules of step (3) with the remaining excipients in a mixer to obtain the final mixture;
(5) tableting the granules of step (3) or the final mixture of step (4) by compressing it on a suitable tablet press to produce the tablet cores; and
(6) optionally film-coating of the tablet cores of step (5) with a film coat.

The invention further provides a method of treating schizophrenia, bipolar disorder or senile dementia in patient comprising administering to said subject a pharmaceutical composition comprises lurasidone or salts thereof, at least one acid and one or more pharmaceutical excipients.

The pharmaceutical compositions described herein are bioequivalent to Latuda®, marketed form of Lurasidone hydrochloride tablets.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1—

TABLE 1A
A. Preparation of Film coated tablets with citric acid

| Sr. No. | Ingredients | Quantity (% w/w) |
|---|---|---|
| 1 | Lurasidone hydrochloride | 14.91 |
| 2 | Microcrystalline cellulose | 38.57 |
| 3 | Pregelatinized starch | 21.12 |
| 4 | Hypromellose | 0.69 |
| 5 | Croscarmellose sodium | 16.15 |
| 6 | Citric acid | 4.97 |
| 7 | Water | q.s. |

Lubrication

| | | |
|---|---|---|
| 8 | Magnesium stearate | 1.24 |

Film Coating

| | | |
|---|---|---|
| 9 | Opadry | 2.36 |
| 10 | Water | q.s. |

Procedure:

Mixture of lurasidone hydrochloride, pregelatinized starch, microcrystalline cellulose and croscarmellose sodium was prepared. Hypromellose and citric acid were dissolved in water and that solution was used to granulate the mixture. The granules were dried and compressed to form tablets. Tablets were then film coated using opadry solution.

TABLE 1B
B. Preparation of Film coated tablets without citric acid

| Sr. No. | Ingredients | Quantity (% w/w) |
|---|---|---|
| 1 | Lurasidone hydrochloride | 14.91 |
| 2 | Microcrystalline cellulose | 38.57 |
| 3 | Pregelatinized starch | 21.12 |
| 4 | Hypromellose | 0.69 |
| 5 | Croscarmellose sodium | 16.15 |
| 7 | Water | q.s. |

Lubrication

| | | |
|---|---|---|
| 8 | Magnesium stearate | 1.24 |

Film Coating

| | | |
|---|---|---|
| 9 | Opadry | 2.44 |
| 10 | Water | q.s. |

Procedure:

Mixture of lurasidone hydrochloride, pregelatinized starch and microcrystalline cellulose, croscarmellose sodium was prepared. This mixture was granulated with aqueous solution of hypromellose. The granules were dried and compressed to form tablets. The tablets were then film coated using opadry solution.

### C. Dissolution Test:

Film coated tablets obtained by above methods were subjected to the dissolution test according to United States Pharmacopoeia, Dissolution apparatus 2, under following conditions:

Dissolution Medium: OGD media (pH 3.8 Mcllvaine buffer)

Paddle rotation: 50 rpm

Volume of the medium: 900 ml

The results of dissolution test are shown below:

Dissolution (% of drug dissolved)

| Time (Min) | Example 1A | Example 1B |
|---|---|---|
| 0 | 0 | 0 |
| 5 | 81 | 76 |
| 10 | 93.8 | 85 |
| 15 | 96.6 | 88.4 |
| 20 | 98.9 | 90.2 |
| 30 | 100.4 | 91.8 |
| 45 | 101.3 | 94.2 |
| 60 | 101.7 | 95.5 |

As shown in the above dissolution results, % of drug dissolved from the tablets is much faster in case of tablets with citric acid as compared to tablets without citric acid.

### Example 2—

TABLE 2
A. Preparation of film coated tablets with citric acid and poloxamer

| Sr. No. | Ingredients | Quantity (% w/w) |
|---|---|---|
| 1 | Lurasidone hydrochloride | 23 |
| 2 | Lactose monohydrate | 7.6 |
| 3 | Microcrystalline cellulose | 33.86 |
| 4 | Pregelatinized starch | 6.7 |
| 5 | Hypromellose | 1.14 |
| 6 | Croscarmellose sodium | 16 |
| 7 | Citric acid | 5.8 |
| 8 | Poloxamer | 2.3 |
| 9 | Water | q.s. |

Lubrication

| | | |
|---|---|---|
| 10 | Magnesium Stearate | 1.16 |

Film Coating

| | | |
|---|---|---|
| 11 | Opadry II | 2.44 |
| 11 | Water | q.s. |

Procedure:

Mixture of lurasidone hydrochloride, lactose, pregelatinized starch, microcrystalline cellulose and croscarmellose sodium was prepared. Hypromellose, poloxamer and citric acid were dissolved in water and that solution was used to granulate the mixture. The granules were dried, lubricated with magnesium stearate and then compressed to form tablets. The tablets were then film coated using opadry II.

### B. Dissolution Test:

Film coated tablets obtained by above methods were subjected to the dissolution test according to United States Pharmacopoeia, Dissolution apparatus 2, under following conditions:

Dissolution Medium: OGD media (pH 3.8 Mcllvaine buffer)

Paddle rotation: 50 rpm

Volume of the medium: 900 ml

The results of dissolution test are shown below:

Dissolution (% of drug dissolved)

| | Reference | |
|---|---|---|
| Time (Min) | (LATUDA) | Example 2A |
| 0 | 0 | 0 |
| 5 | 51.2 | 57.4 |
| 10 | 86.2 | 94.7 |
| 15 | 90.7 | 97.7 |
| 20 | 92.5 | 99.2 |
| 30 | 94.7 | 99.6 |

As shown in the above dissolution results, % of drug dissolved from the tablets prepared according to present invention is comparable to LATUDA (marketed brand of lurasidone hydrochloride in USA).

1. A pharmaceutical composition comprising Lurasidone or pharmaceutically acceptable salts thereof, at least one acid, and one or more pharmaceutically acceptable excipients.

2. The pharmaceutical composition according to claim 1, wherein the acid is an organic acid and/or an inorganic acid.

3. The pharmaceutical composition according to claim 1, wherein the acid is present in an amount of 3-7% of the total weight of composition.

4. The pharmaceutical composition according to claim 2, wherein the organic acid comprises one or more of citric acid, malic acid, fumaric acid, tartaric acid, succinic acid, ascorbic acid and hydrates thereof.

5. The pharmaceutical composition according to claim 2, wherein the inorganic acid comprises one or more of hydrochloric acid, boric acid, sulfuric acid, and phosphoric acid.

6. The pharmaceutical composition according to claim 1, wherein the composition is in a form suitable to provide immediate release of lurasidone or salts thereof.

7. The pharmaceutical composition according to claim 1, further comprising at least one surfactant.

8. The composition according to claim 1, wherein the pharmaceutically acceptable excipient is selected from a diluent, a disintegrant, a binder, a stabilizer, a buffering agent, a lubricant, a glidant, an antiadherent, a solubilizer, a sweetener, a flavoring agent or a solvent.

9. A pharmaceutical composition comprising
(a) about 5-45% of Lurasidone or pharmaceutically acceptable salts thereof,
(b) about 0.1-10% of at least one acid,
(d) about 0.5-10% of at least one surfactant.

10. The pharmaceutical composition according to claim 9, wherein the surfactant comprises one or more of polyoxyethylene glycol sorbitan alkyl esters, sorbitan alkyl esters, glycerol alkyl esters, glucoside alkyl ethers, copolymers of polyalkyleneglycols, sodium lauryl sulfate.

11. The composition according to claim 1, wherein the composition retains at least 80% of the potency of lurasidone in the said composition after storage for three months at 40° C. and 75% relative humidity.

12. A process of preparing pharmaceutical composition according to claim 1, comprising
i. mixing and/or granulating lurasidone, at least one acid and one or more pharmaceutically acceptable excipients;
ii. compressing the mixture or granules to form a tablet; and
iii. optionally coating the tablet.

13. A method of treating schizophrenia, bipolar disorder or senile dementia, the method comprising administering to a human patient in need thereof the pharmaceutical composition of claim 1.
